(19) United States
(12) Patent Application Publication
(10) Pub. No.: US 2021/0113140 A1
(43) Pub. Date: Apr. 22, 2021

(54) CONCUSSION AND SUB-CONCUSSION MONITORING SYSTEM AND METHOD

(71) Applicant: Jason Stanley McEwen, Victoria (CA)

(72) Inventor: Jason Stanley McEwen, Victoria (CA)

(21) Appl. No.: 16/659,980

(22) Filed: Oct. 22, 2019

(51) Int. Cl. A61B 5/00; A61B 5/024; G06F 17/18

(52) U.S. Cl. CPC A61B 5/4035; G06F 17/18; A61B 5/0022; A61B 5/02405

(57) ABSTRACT

A method for determining a state of concussion or sub-concussion of an individual by measuring using a monitoring device a plurality of baseline heart rate variability data over a first period of time. The monitoring device transmits said baseline heart rate variability (HRV) data to an application on a smart device. The baseline HRV data is transmitted by said smart device said baseline heart rate variability data to a memory. The monitoring device measures a plurality of contact activity heart rate variability data over a second period of time and transmits the contact activity heart rate variability data to the smart device. The smart device transmits said contact activity heart rate variability data to a memory. A processor analyzes the plurality of baseline heart rate variability data and the plurality of contact activity heart rate variability data in the memory to determine a state of autonomic nervous system dysfunction by comparing the contact activity heart rate variability data with the baseline heart rate variability and communicates said state to said individual via said application on said smart device.

### BACKGROUND

Traumatic brain injuries (TBI) are classified as being either mild, moderate, or severe. High-impact collisions from sport or from motor vehicle crashes produce moderate to severe TBI that are observable using current imaging techniques such as x-ray computed tomography (CT) or magnetic resonance imaging (MRI). Recently, researches are becoming increasingly aware of the brain damage that results from repetitive sub-concussive head impacts (RSHI) sustained in the contact sport arena [1] or on the battlefield from IED blasts. The resulting mild TBI (mTBI) is not readily observable by traditional CT or MRI scans. The cumulative effects of RSHI generate shearing of axonal projections [2]. However, this chronic damage will most often not manifest to become a clinically relevant diagnosis of concussion. In the long-term, repetitive sub-concussive head impacts (RSHI) can lead to chronic traumatic encephalopathy (CTE) [3].

The brain and nervous system control the beating of the heart. When the body requires an increased heart rate, during exercise for example, signals are sent to the heart signaling a rate increase via the sympathetic branch (SNS) of the autonomic nervous system (ANS). When the body requires a decreased heart rate during rest or sleep, the parasympathetic branch (PNS) of the autonomic nervous system, through the vagus nerve, signals the heart to slow down. There is continuous interplay between the SNS and PNS. Sympathetic dominance results in a faster heart rate (HR) while parasympathetic dominance results in a slower heart rate. Heart rate is an average, the amount of heart beats during an interval of one minute. This is a crude measurement that excludes a wealth of information regarding the subtle interplay between the SNS and PNS. This subtle interplay between the SNS and PNS is observable through accurate measurement of the RR interval (also referred to as Inter-Beat Interval or IBI) and is termed Heart Rate Variability (HRV).

HRV has been gaining interest from many health fields in recent times including the cardiac, exercise, and sleep sciences. HRV is a valuable method for determining the health and function of an individual's ANS because HRV can be measured directly and objectively. HRV is influenced by a multitude of factors and tends to fluctuate over the course of the day, week, month or year. HRV is best measured during sleep, rest, or during light exercise.

HRV is mainly determined by the amount of input the heart receives from the PNS. A healthy or normal functioning PNS results in a higher HRV value while a stressed, damaged, or dysfunctional PNS results in a lower HRV value. There are two modalities by which HRV can be analyzed. Time domain statistics includes the parameters SDNN, SENN, SDSD, RMSSD, NN50, and pNN50, with RR triangular index, and TINN belonging to the geometrical class of parameters. Frequency domain statistics consists of Low Frequency (LF), Very Low Frequency (VLF), and High Frequency (HF) parameters. The LF band is from 0.04-0.15 Hz, the VLF band is from 0.0033-0.04 Hz, and the HF band is from 0.15-0.4 Hz. The LF and VLF bands represent the contribution of the SNS to HRV while the HF band represents the contribution of the PNS to HRV. Frequency domain analysis of the complete HR spectrum provides the ability to discriminate between the SNS and PNS contribution to HRV.

The many psychological and physiological forces that regulate HRV contribute to the complexity of the IBI Interval. Stress and illness, demographics such as age and gender, as well as exercise habits can all influence HRV. In order for HRV changes to be detected accurately, proper control data must be utilized. Considering the inherent variance in HRV that exists between individuals as well as the fluctuating HRV values within individuals, using the proper control data for comparison to experimental data is imperative to obtaining accurate results. To establish baseline control data, a period of collection prior to the engagement in contact sport is required. Once well-established baseline data has been captured for sleep, rest, and exercise states, then the individual is cleared for any activity in which repetitive head contacts or sub-concussive blasts might be experienced. Recent studies have shown that HF HRV gradually increases post-concussion [6][7]. Should the individual sustain a clinically relevant concussion during contact sport or military deployment, then post-concussion HRV data can be compared to the pre-activity baseline data to aid in the decision to return the individual to activity.

In order to collect the required background data as well as record ongoing subtle damage to the brain that will occur during contact sport or military deployment, a realistic method of detection needs to be developed. Studies have shown that high powered diagnostic imaging equipment is capable of detecting axonal shearing due to RSHI. However, this method is expensive, time-consuming, and requires the presence of highly trained medical personal. It is just not feasible to use current methods to analyze brain damage due to RSHI in large numbers of people, performing multiple tests per week, for prolonged periods of time.

There is a need for a device that has the ability to collect, store, and compare the HRV data required for the purpose of detecting onset of brain damage caused by RSHI. There is also a need for a device to provide information on the recovery of brain damage resulting from a clinically diagnosed concussion. There is a need for a device to detect very subtle changes in HRV prior to there ever being a concussion. HRV changes occur cumulatively as the brain is damaged from repetitive head contacts experienced in sport or military deployment. There is a need for an individual to obtain baseline HRV and test HRV data without any need to visit a member of the medical personnel or hospital/clinic.

References:

[1] Hirad et al., Sci. Adv. 2019; 5: eaau3460 7 Aug. 2019. https://advances.sciencemag.org/content/5/8/eaau3460

[2] Subconcussive Head Trauma. William A. Cox, Forensic Science Newsletter, Nov. 1, 2016. https://pdfs.semanticscholar.org/c324/33b6d353df713f5e734c009f7209a20d0b68.pdf

[3] Long-term Consequences of Repetitive Brain Trauma: Chronic Traumatic Encephalopathy. Robert A. Stern, 26 Oct. 2011, Concussion and Mild Traumatic Brain Injury: Current and Future Concepts

[4] Heart rate variability in normal and pathological sleep : Eleonora Tobaldini, Front Physiol. 2013; 4: 294.

[5] Modulation of the Sympatho-Vagal Balance during Sleep: Frequency Domain Study of Heart Rate Variability and Respiration. Ramona Cabiddu, Front Physiol. 2012; 3: 45.

[6] Heart rate variability following youth concussion: how do autonomic regulation and concussion symptoms differ over time postinjury? Paniccia M, BMJ Open Sport & Exercise Medicine 2018; 4: e000355. doi:10.1136/bmjsem-2018-000355

[7] Severity of traumatic brain injury correlates with long-term cardiovascular autonomic dysfunction. Max J. Hilz, J Neurol (2017) 264:1956-1967

### BRIEF SUMMARY

A method for determining a state of concussion or sub-concussion of an individual by measuring using a monitoring device a plurality of baseline HRV data over a first period of time. The monitoring device transmits said baseline HRV data to an application on a smart device. The baseline HRV data is transmitted by said smart device said baseline heart rate variability data to a memory. The monitoring device measures a plurality of contact activity heart rate variability data over a second period of time and transmits the contact activity heart rate variability data to the smart device. The smart device transmits said contact activity heart rate variability data to a memory. A processor analyzes the plurality of baseline heart rate variability data and the plurality of contact activity heart rate variability data in the memory to determine a state of autonomic nervous system dysfunction by comparing the contact activity heart rate variability data with the baseline heart rate variability and communicates said state to said individual via said application on said smart device.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a high-level system diagram 100 in accordance with one embodiment.

FIG. 2 illustrates a baseline workflow 200 in accordance with one embodiment.

FIG. 3 illustrates an active workflow 300 in accordance with one embodiment.

FIG. 4 illustrates a 1-year measurement timeline 400 in accordance with one embodiment.

### DETAILED DESCRIPTION

The details of one or more embodiments of the subject matter of this specification are set forth in the accompanying drawings and the description below. Other features, aspects, and advantages of the subject matter will become apparent from the description, the drawings, and the claims.

Like reference numbers and designations in the various drawings indicate like elements.

During sleep, the human body systems cycle through wake, non-REM, and REM stages. In the initial transition from wakefulness to the first non-REM sleeping stage, the PNS takes control over the SNS and the HF band of HRV is significantly increased. After an initial period in non-REM sleep, the SNS takes control causing a transition to REM sleep. Multiple times over the course of a 5-8 hour long sleep period our body will alternate between non-REM and REM sleep stages producing the sleep cycle. In each stage, timed intervals (usually 5 minutes) are recorded with subsequent statistical analysis. HRV is higher during the non-REM stages and lower during the REM sleep stages. More specifically, the HF component of HRV will be higher during non-REM stages and lower during REM stages [4] [5]. A major factor for the higher HF measurement during the non-REM sleep stages is due to the phenomenon of Respiratory Sinus Arrhythmia (RSA).

The cardiac Parasympathetic Nervous System (PNS) consists of pre-ganglionic nerve cells that have cell bodies originating mainly in the nucleus ambiguous of the lower brainstem. These pre-ganglionic cells connect to post-ganglionic cells that innervate the sinus node and atrioventricular node of the heart. The brainstem is a significant area of white matter injury during rotational Repetitive Sub-concussive Head Impacts (RSHI) [1]. Should the area of the brainstem that contains the PNS pre-ganglionic nerve cell bodies become injured during RSHI or from combat blasts, a decrease in HRV is expected. Conversely, if changes to HRV is detected during the time of contact sport or military deployment, then those HRV changes would be attributable to damage to the brain.

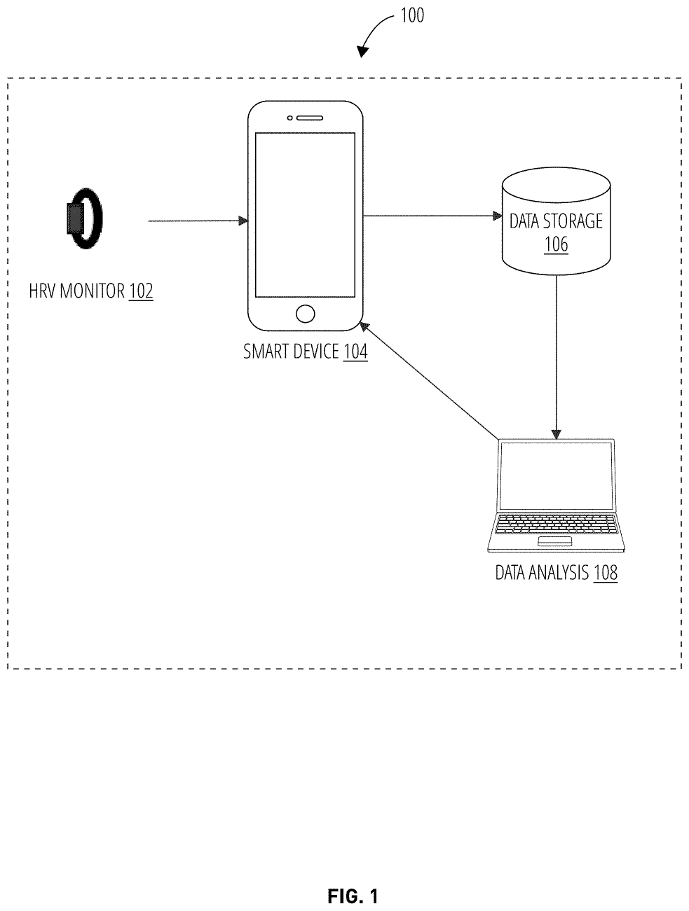

FIG. 1 provides a high-level system diagram 100 of an embodiment of the system. The individual's HRV is measured using and HRV monitor 102. The monitoring is done during periods of contact activity and inactivity. The contact activity is defined as a period of time where the individual participates in activities involving potential impact(s) to their head, such as, but not limited to, playing hockey or football or military exercises. The inactivity period is defined as a period of time where the individual does not participate in activities involving potential impact(s) to their head, where baseline measurements are established.

The HRV monitor 102 can be worn on the wrist, chest, arm, finger, feet or ear. Other types of HRV monitor 102 such as mattress pads in close proximity to the individual can also be used as long as the individual is not bothered by the device while sleeping. The HRV monitor 102 can include an electrocardiogram (ECG) or use photoplethysmography (PPG) technology. Any type of monitor using any type of technology known in the art capable of measuring HRV and communicating to a smart device 104 a can be used.

The individual uses the HRV monitor 102 and the application on the smart device 104, prior to commencing in contact sport or military deployment. For a predetermined number of nights (preferably at least 15), the HRV monitor 102 is worn every night while sleeping. For each night, the baseline sleep HRV data is collected during the sleep hours (5-8 hours). The HRV monitor 102 is installed on the individual as per the HRV monitor 102 instructions prior to sleep and paired with the smart device 104. The application detects the commencement of sleep by the changes in HRV during the initial transition from wakefulness to sleeping state. The HRV data from all the nights is then an average is calculated to become the baseline sleep HRV data. During the baseline sleep HRV data collection period the individual cannot participate in any activity where there is a chance of sustaining forces to the head/brain from contact or abrupt acceleration. There could also be a questionnaire filed by the individual explaining any such sustained forces that might have been beyond control such as car crash, falling off bike, etc., during the baseline collection phase.

Optionally, during the period prior to contact activity when the baseline sleep HRV Data is being collected, a baseline exercise HRV Data can also be collected. The individual engages in all the physical activities they normally would and wears the HRV monitor 102 during these activities. These activities should not involve head contact sports, but most training activities such as running, cycling, weight training, are encouraged. During exercise, any HRV data generated while the heart rate is above a certain threshold, is used for the baseline exercise HRV Data.

Baseline HRV data is transmitted from the HRV monitor 102 via Bluetooth, or any other wireless data exchanging technology known in the art, to an application on a smart device 104, such as, but not limited to, a mobile phone device that is capable of ensuring proper user identification through secure biometric or password protection.

When the individual starts a contact activity period, the individual notifies the application on the smart device 104. The individual continues to use the HRV monitor 102 during sleep (and optionally non-contact exercise) to gather active sleep HRV data (and optionally active exercise HRV data). The HRV data (baseline and contact activity) is analyzed 108 to determine whether there has been damage to the brain. The determination is conveyed to the individual via the smart device. The baseline data serves as a control for each individual and makes it possible to extract the subtle changes in active sleep HRV data compared with baseline sleep HRV data arising from repetitive forces to the head during the contact activity period. Optionally, subtle changes in active exercise HRV data compared with baseline exercise HRV data arising from repetitive forces to the head during the contact activity period. When changes are detected, the application notifies the individual of positive or negative changes. The application can also be configured to notify a coach or medical staff of the changes.

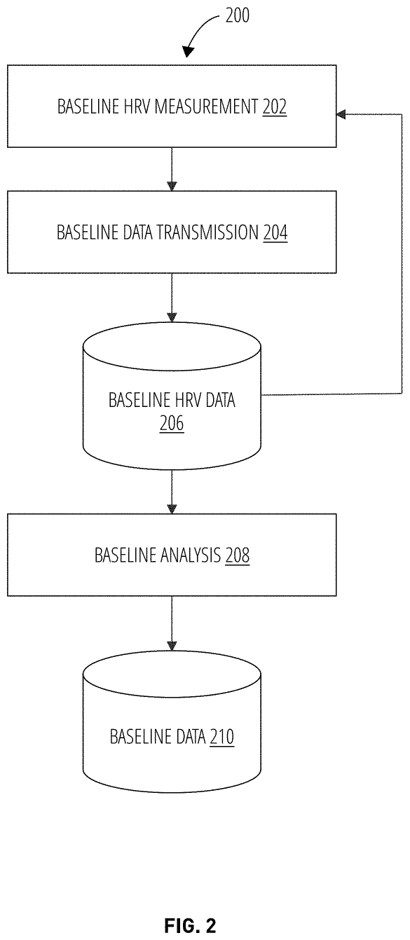

FIG. 2 provides an overview of the baseline workflow 200 as used in one embodiment of the system.

In block 202, the baseline HRV measurement is performed on an individual during a period of contact inactivity. Preferably, the measurements are performed while the individual is sleeping and optionally during exercise as described above. The inter-beat interval (IBI) data is recorded by the HRV monitor 102, and the IBI data is converted into binary code. The IBI binary code data collected by the HRV monitor 102 is transmitted 204 via Bluetooth (or something similar) to the individual's smart device 104 with the aid of a secure application. All binary code IBI data is relayed from the smart device 104 is stored 206 in a data storage 106 (possibly but not limited to cloud storage). The binary code IBI data are retrieved from the data storage facility and a baseline analysis 208 is performed and stored 210 in the data storage 106 for later use.

The baseline analysis 208 includes a series of preprocessing steps, including but not limited to, sampling and digitizing, artifact identification, IBI data editing, IBI interval rejection, IBI data sequencing. Most commonly, power spectral analysis of HRV is analyzed through fast Fourier transform and autoregressive models, by commercial devices or non-commercial software known in the art. Commonly HRV is analyzed by four methods; time-domain, frequency-domain, time- frequency domain and non-linear methods. Any other methods known in the art for analyzing HRV can be used.

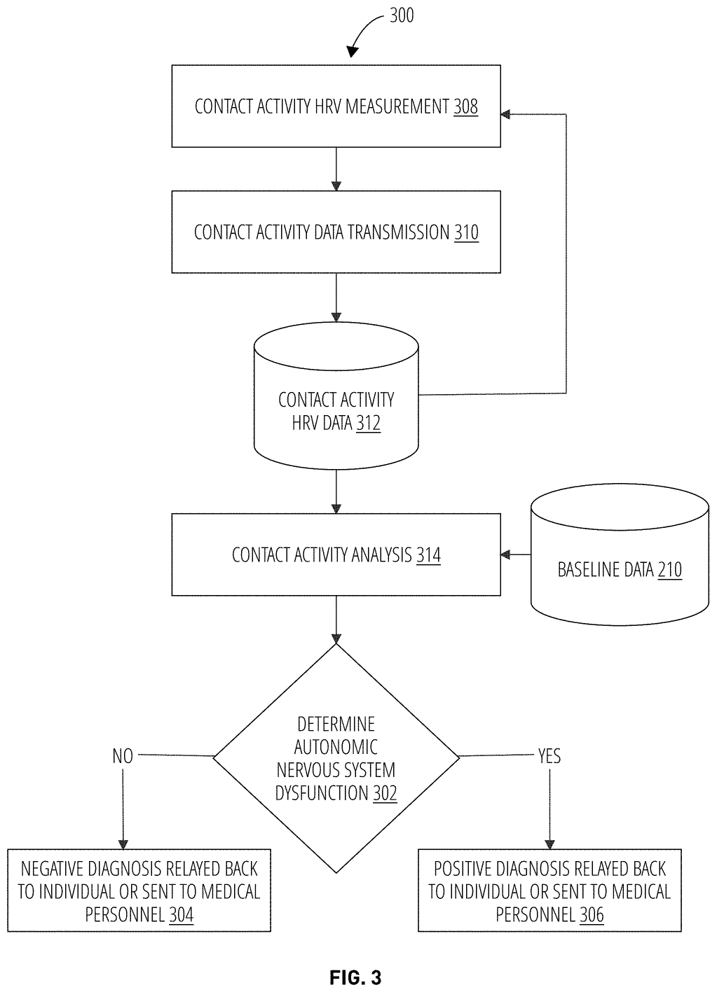

FIG. 3 provides an overview of the active workflow 300 as used in one embodiment of the system.

In block 308, the contact activity HRV measurement is performed on an individual during a period of contact activity. Preferably, the measurements are performed while the individual is sleeping. Optionally, other periods of measurements can also be used such as during light exercise as described above.

In block 310, the contact activity data is transmitted from the HRV monitor 102 to a smart device 104.

In block 312, the contact activity HRV data is stored in the contact activity HRV data memory. Additional contact activity HRV data may be acquired by returning to block 202.

In block 314, the contact activity HRV analysis is performed using contact activity sleep HRV data, baseline sleep HRV data and the HRV analysis software. Any HRV algorithm known in the art can be used for the analysis.

In decision block 302, the determination of potential autonomic nervous system dysfunction is performed from the contact activity analysis. For example, if the contact activity sleep HRV is lower than the baseline sleep HRV by a predetermined threshold then the application detects potential damage to the brain. Optionally, if the contact activity exercise HRV is lower than the baseline sleep HRV by a predetermined threshold then the application detects potential damage to the brain. Other comparisons can be established to compare the baseline HRV data (sleep or exercise) with the contact activity HRV data to determine autonomic nervous system dysfunction 302, for example, but not limited to, using a dynamically adjusted threshold, a threshold configured in the application.

In block 304, a potential negative diagnosis may be relayed back to individual or sent to medical personnel.

In block 306, a potential positive diagnosis may be relayed back to individual or sent to medical personnel.

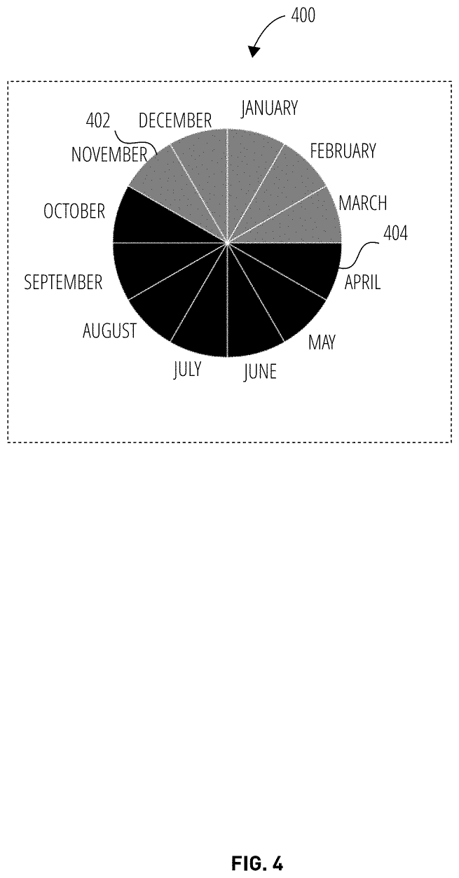

FIG. 4 illustrates a 1-year measurement timeline 400 for an individual who engages in a contact activity for specific months of the year. In this example, the individual has an active period 404, the period of time where the individual plays soccer, between April and October and an inactive period 402, the period of time where the individual does not play soccer, between November and March. The baseline HRV and contact activity HRV are measured and analyzed throughout the inactive period 402 and active period 404 respectively. A comparison of the baseline HRV and contact activity HRV analyses is then utilized in the determination of the potential onset or progression of minor traumatic brain injuries which is delivered to the individual or to medical personnel.

A computer program (which may also be referred to or described as a software application, code, a program, a script, software, a module or a software module) can be written in any form of programming language. This includes compiled or interpreted languages, or declarative or procedural languages. A computer program can be deployed in many forms, including as a module, a subroutine, a standalone program, a component, or other unit suitable for use in a computing environment. A computer program can be deployed to be executed on one computer or can be deployed on multiple computers that are located at one site or distributed across multiple sites and interconnected by a communication network.

Computers suitable for the execution of a computer program include, by way of example, general or special purpose microprocessors or both, or any other kind of central processing unit. Generally, a central processing unit receives instructions and data from a read-only memory or a random-access memory or both. A computer can also include, or be operatively coupled to receive data from, or transfer data to, or both, one or more mass storage devices for storing data, e.g., optical disks, magnetic, or magneto optical disks. It should be noted that a computer does not require these devices. Furthermore, a computer can be embedded in another device. Non-limiting examples of the latter include a game console, a mobile telephone a mobile audio player, a personal digital assistant (PDA), a video player, a Global Positioning System (GPS) receiver, or a portable storage device. A non-limiting example of a storage device include a universal serial bus (USB) flash drive.

Computer readable media suitable for storing computer program instructions and data include all forms of non-volatile memory, media and memory devices; non-limiting examples include magneto optical disks; semiconductor memory devices (e.g., EPROM, EEPROM, and flash memory devices); CD ROM disks; magnetic disks (e.g., internal hard disks or removable disks); and DVD-ROM disks. The processor and the memory can be supplemented by, or incorporated in, special purpose logic circuitry.

To provide for interaction with a user, embodiments of the subject matter described herein can be implemented on a computer having a display device for displaying information to the user and input devices by which the user can provide input to the computer (e.g., a keyboard, a pointing device such as a mouse or a trackball, etc.). Other kinds of devices can be used to provide for interaction with a user. Feedback provided to the user can include sensory feedback (e.g., visual feedback, auditory feedback, or tactile feedback). Input from the user can be received in any form, including acoustic, speech, or tactile input. Furthermore, there can be interaction between a user and a computer by way of exchange of documents between the computer and a device used by the user. As an example, a computer can send web pages to a web browser on a user's client device in response to requests received from the web browser.

Embodiments of the subject matter described in this specification can be implemented in a computing system that includes: a front end component (e.g., a client computer having a graphical user interface or a Web browser through which a user can interact with an implementation of the subject matter described herein); or a middleware component (e.g., an application server); or a back end component (e.g. a data server); or any combination of one or more such back end, middleware, or front end components. The components of the system can be interconnected by any form or medium of digital data communication, e.g., a communication network. Non-limiting examples of communication networks include a local area network (“LAN”) and a wide area network (“WAN”).

The computing system can include clients and servers. A client and server are generally remote from each other and typically interact through a communication network. The relationship of client and server arises by virtue of computer programs running on the respective computers and having a client-server relationship to each other.

While operations are depicted in the drawings in a particular order, this should not be understood as requiring that such operations be performed in the particular order shown or in sequential order.

What is claimed is:

1. A method for determining a state of concussion or sub-concussion of an individual, the method comprising:
measuring using a monitoring device coupled to said individual a plurality of baseline heart rate variability data over a first period of time, wherein said monitoring device transmits said baseline heart rate variability data to an application on a smart device coupled to said monitoring device;
transmitting by said smart device said baseline heart rate variability data to a memory;
measuring using said monitoring device a plurality of contact activity heart rate variability data over a second period of time, wherein said monitoring device transmits the contact activity heart rate variability data to said smart device;
transmitting by said smart device said contact activity heart rate variability data to said memory;
analyzing at a processor coupled to said smart device and to said memory said plurality of baseline heart rate variability data and said plurality of contact activity heart rate variability data;
determining by said processor a state of autonomic nervous system dysfunction by comparing said contact activity heart rate variability data with said baseline heart rate variability;
communicating said state to said individual via said application on said smart device.

2. The method of claim 1 wherein said monitoring device in contact with said individual's body.

3. The method of claim 1 wherein said monitoring device is within close proximity to said individual's body.

4. The method of claim 1 wherein said measuring comprises:
measuring a plurality of inter-beat intervals over a predetermined duration of time;
calculating an average of said plurality of inter-beat intervals.

5. The method of claim 4 wherein said predetermined duration of time is at least 5 minutes.

6. he method of claim 1 wherein the number of measurements of said baseline heart rate variability data is at least 15.

7. A system for determining a state of concussion or sub-concussion of an individual, the system comprising:
a monitoring device coupled to said individual to monitor a plurality of baseline heart rate variability data over a first period of time, wherein said monitoring device transmits said baseline heart rate variability data to an application on a smart device coupled to said monitoring device;
said smart device transmits said baseline heart rate variability data to a memory;
said monitoring device measures a plurality of contact activity heart rate variability data over a second period of time, wherein said monitoring device transmits the contact activity heart rate variability data to said smart device;
said smart device transmits said contact activity heart rate variability data to said memory;
a processor coupled to said smart device and to said memory to analyze said plurality of baseline heart rate variability data and said plurality of contact activity heart rate variability data;
said processor determines a state of autonomic nervous system dysfunction by comparing said contact activity heart rate variability data with said baseline heart rate variability and communicates said state to said individual via said application on said smart device.

8. The system of claim 7 wherein said monitoring device in contact with said individual's body.

9. The system of claim 7 wherein said monitoring device is within close proximity to said individual's body.

10. The system of claim 7 wherein said monitoring device measures a plurality of inter-beat intervals over a predetermined duration of time and calculates an average of said plurality of inter-beat intervals.

11. The system of claim 7 wherein said predetermined duration of time is at least 5 minutes.

12. The system of claim 7 wherein the number of measurements of said baseline heart rate variability data is at least 15.
